(19) United States
(12) Patent Application Publication
(10) Pub. No.: US 2014/0121580 A1
(43) Pub. Date: May 1, 2014

(54) DRESSING

(71) Applicant: TATUNG COMPANY, Taipei (TW)

(72) Inventor: Cheng-Yang Lee, Taipei (TW)

(73) Assignee: TATUNG COMPANY, Taipei (TW)

(21) Appl. No.: 13/772,368

(22) Filed: Feb. 21, 2013

(30) Foreign Application Priority Data
Nov. 1, 2012 (TW) 101221182

(51) Int. Cl. A61F 13/02

(52) U.S. Cl. CPC A61F 13/0213
USPC 602/43

(57) ABSTRACT

A dressing is provided. The dressing includes a back layer, a composite substrate, and a gel layer. The back layer has a surface. The composite substrate is disposed on the surface of the back layer, and includes a substrate layer and at least one barrier layer. The barrier layer is disposed between the back layer and the substrate layer. The gel layer is disposed on the composite substrate, wherein the substrate layer is located between the gel layer and the barrier layer. The barrier layer prevents liquid from permeating from the substrate layer to the surface of the back layer.

### CROSS-REFERENCE TO RELATED APPLICATION

This application claims the priority benefit of Taiwan application serial no. 101221182, filed on Nov. 1, 2012. The entirety of the above-mentioned patent application is hereby incorporated by reference herein and made a part of this specification.

### BACKGROUND OF THE INVENTION

1. Field of the Invention

The invention relates to a dressing, and more particularly, to a dressing having a composite substrate.

2. Description of Related Art

The fabrication process of a dressing usually includes the following steps. First, the main material of the dressing is coated on a substrate layer to react. Then, the process is finished by cropping, posting, stamping, and packaging according to the desired size. In particular, since the main material is usually a runny liquid and the substrate layer is a porous material, therefore, when coating the main material, the main material is embedded in the cavities of the substrate layer to facilitate physical adhesion and polymerization at the same time in order to complete the fabrication of gels such as hydrogel. However, when coating the main material, the unreacted main material may permeate through the cavities of the substrate layer to the back of the substrate layer, which may probably cause the main material to not react completely and raise security concerns, and also causing the formed main material layer have defects such as an inconsistent thickness.

Over time, the unreacted main material may diffuse through the bottom of the substrate layer to the reacted main material. Therefore, skin irritation, allergy, or other conditions in the wound may result when the dressing contacts the skin. Moreover, since the substrate layer is usually a porous material, the contact area between the substrate layer and the back layer is small when adhering. Therefore, the adhesion between the substrate layer and the back layer is reduced when a main material such as hydrogel absorbs wound exudates and the exudates diffuse in the main material. In this way, the substrate layer may fall off of the back layer when the dressing is replaced, causing inconvenience in use.

### SUMMARY OF THE INVENTION

The invention provides a dressing including a composite substrate, wherein the barrier layer may prevent liquid from permeating from the substrate layer to the surface of the back layer.

The invention provides a dressing including a back layer, a composite substrate, and a gel layer. The back layer has a surface. The composite substrate is disposed on the surface of the back layer, and includes a substrate layer and at least one barrier layer. The barrier layer is disposed between the back layer and the substrate layer. The gel layer is disposed on the composite substrate, wherein the substrate layer is located between the gel layer and the barrier layer. The barrier layer prevents liquid from permeating from the substrate layer to the surface of the back layer.

In an embodiment of the invention, the composite substrate includes an adhesive layer disposed between the barrier layer and the substrate layer.

In an embodiment of the invention, the adhesive layer includes a hot melt adhesive, a pressure sensitive adhesive, or an acrylic glue.

In an embodiment of the invention, the barrier layer includes a lamination layer.

In an embodiment of the invention, the gel layer includes a hydrogel layer.

In an embodiment of the invention, the substrate layer includes a porous membrane.

In an embodiment of the invention, the material of the substrate layer includes polyethylene, polypropylene, polyester, polycarbonate, polyurethanes, the derivatives thereof, or a combination thereof.

In an embodiment of the invention, the barrier layer includes a non-porous membrane.

In an embodiment of the invention, the material of the barrier layer includes polyethylene, polypropylene, polyester, polycarbonate, polyurethanes, the derivatives thereof, or a combination thereof.

In summary, the invention provides a dressing having a composite substrate, wherein the composite substrate includes a substrate layer and a barrier layer, and the barrier layer may prevent liquid from permeating from the substrate layer to the surface of the back layer. In this way, permeation of the solution to the surface of the back layer may be avoided, and thus the ingredients in the solution coated on the composite substrate can be reacted completely, to form a gel layer with a uniform thickness. Accordingly, the adhesion between the composite substrate and the back layer is improved. Therefore, the safety, the stability, and the convenience of use of the dressing are significantly improved.

In order to make the aforementioned and other features and advantages of the invention more comprehensible, several embodiments accompanied with figures are described in detail below.

### BRIEF DESCRIPTION OF THE DRAWINGS

The accompanying drawings are included to provide a further understanding of the invention, and are incorporated in and constitute a part of the specification. The drawings illustrate embodiments of the invention and, together with the description, serve to explain the principles of the invention.

FIG. 1 is an exploded view of a dressing according to an embodiment of the invention.

FIG. 2 is an exploded view of a dressing according to an embodiment of the invention.

### DESCRIPTION OF THE EMBODIMENTS

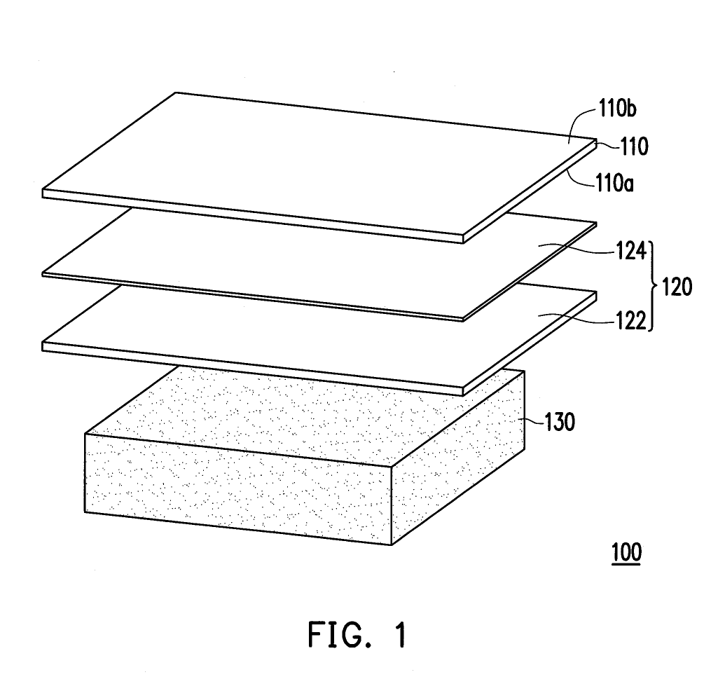

FIG. 1 is an exploded view of a dressing according to an embodiment of the invention. Referring to FIG. 1, the dressing 100 includes a back layer 110, a composite substrate 120, and a gel layer 130. The back layer 110 has surfaces 110a and 110b. The surface 110a is, for instance, an inner surface contacting the skin, and the surface 110b is, for instance, an exposed outer surface. The material of the back layer 110 is, for instance, polyethylene, polypropylene, polyester, polycarbonate, polyurethanes, the derivatives thereof, or a combination thereof. The main function of the back layer 110 is to provide water resistance for the dressing. In an embodiment, a layer of medical grade pressure sensitive adhesive (not illustrated) is, for instance, coated on the surface 110a of the back layer 110 to provide fixity for the dressing.

The composite substrate 120 is disposed on the surface 110a of the back layer 110 and includes a substrate layer 122 and at least one barrier layer 124. The barrier layer 124 is disposed between the back layer 110 and the substrate layer 122. In the present embodiment, the substrate layer 122 includes, for instance, a porous membrane, and the porous membrane is, for instance, polyethylene, polypropylene, polyester, polycarbonate, polyurethanes, the derivatives thereof, or a combination thereof. The barrier layer 124, for instance, includes a non-porous membrane. In the present embodiment, the barrier layer 124 is, for instance, a lamination layer, and the lamination layer is, for instance, a polymer membrane on the substrate layer 122, wherein the polymer membrane is directly formed by lamination. The material of the polymer membrane includes polyethylene, polypropylene, polyester, polycarbonate, polyurethanes, the derivatives thereof, or a combination thereof. The thickness ratio of the barrier layer 124 to the substrate layer 122 is, for instance, between 0.1 and 0.8, and the ratio of the weight per unit area of the barrier layer 124 to the substrate layer 122 is, for instance, between 0.5 and 3. It should be mentioned that, the material of the substrate layer 122 may be composed of materials such as cotton, hemp, or chemical synthetic fiber, and the substrate layer 122 has interweaving mesh structure and cavities. In other words, both the front of the substrate layer 122 (the surface contacting the gel layer 130) and the back (the surface contacting the barrier layer 124) have cavities, wherein the cavities in the front may improve the adhesion between the gel layer 130 and the substrate layer 122, and the cavities in the back may be covered or filled by the barrier layer 124. Therefore, after the substrate layer 122 and the barrier layer 124 are joined, one side of the substrate layer 122 still has cavities to improve the adhesion with the gel layer 130. However, the cavities on the other side of the substrate layer 122 are covered or filled by the barrier layer 124 to prevent liquid from permeating from the substrate layer 122 to the surface 110a of the back layer 110. For instance, when the barrier layer 124 is formed by lamination, the liquid material used to form the barrier layer 124 is, when solidifying, embedded in the cavities of the substrate layer 122 to fill the cavities. Since the material of the barrier layer 124 is embedded in the cavities of the substrate layer 122, the adhesion between the barrier layer 124 and the substrate layer 122 is improved, and the two become hard to separate. The composite substrate 120 having, in essence, no cavities in the back, is formed after the substrate layer 122 and the barrier layer 124 are joined, however, the adhesion between the substrate layer 122 and the barrier layer 124 may still be individually separated or disassembled by force.

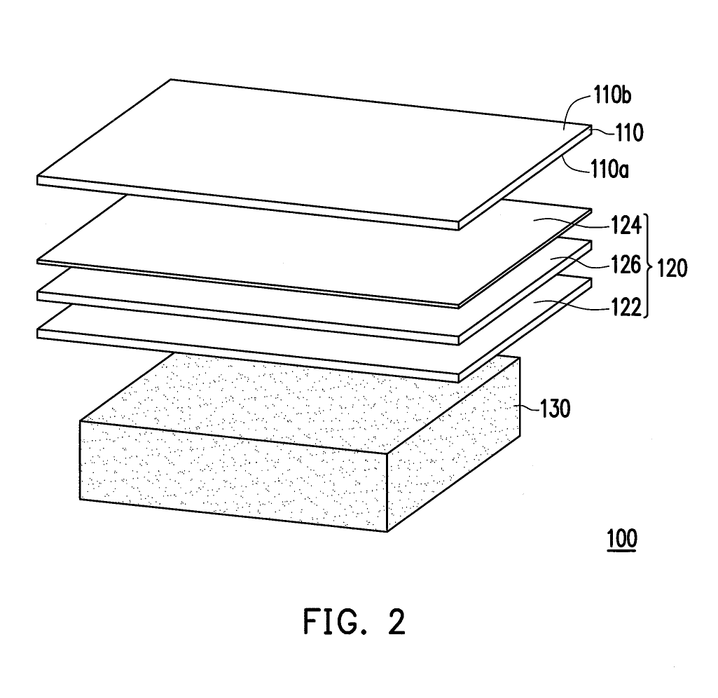

In the present embodiment, the barrier layer 124 is formed directly on the substrate layer 122, but the invention is not limited thereto. In other words, the barrier layer 124 may be joined directly to the substrate layer 122 or joined to the substrate layer 122 by layers such as an adhesive layer. For instance, in another embodiment, as shown in FIG. 2, the composite substrate 120 further includes an adhesive layer 126 disposed between the barrier layer 124 and the substrate layer 122. In detail, an adhesive layer 126 is coated on the barrier layer 124 after the barrier layer 124 is plastic molded or shaped, and the barrier layer 124 is joined to the substrate layer 122 by the adhesive layer 126. In the present embodiment, the front of the substrate layer 122 (the side contacting the gel layer 130) still has cavities to improve the adhesion between the gel layer 130 and the substrate layer 122. The cavities in the back of the substrate layer 122 (the side contacting the adhesive layer 126) may be covered or filled by the adhesive layer 126 and the barrier layer 124 to improve the adhesion between the adhesive layer 126 and the substrate layer 122 and to prevent liquid from permeating from the substrate layer 122 to the surface 110a of the back layer 110. Therefore, the composite substrate 120 having, in essence, no cavities in the back, is formed when the substrate layer 122, the adhesive layer 126, and the barrier layer 124 are joined. In the present embodiment, the cavities of the barrier layer 124 are much smaller or much less than the cavities of the substrate layer 122, and therefore may be treated as non-existent. The material of the barrier layer 124 is, for instance, polyethylene, polypropylene, polyester, polycarbonate, polyurethanes, the derivatives thereof, or a combination thereof The adhesive layer 126 includes, for instance, a hot melt adhesive, a pressure sensitive adhesive, or an acrylic glue. Moreover, in the fabrication process, the barrier layer 124 is first formed on the substrate layer 122 to fabricate the composite substrate 120, and then the composite substrate 120 is joined to the back layer 110. It should be mentioned that, since the barrier layer 124 of the composite substrate 120 has, in essence, a flat surface without cavities, the contact area between the composite substrate 120 and the back layer 110 is significantly increased, resulting in greater adhesion between the composite substrate 120 and the back layer 110. In this way, the separation of the composite substrate 120 from the back layer 110 when the dressing 100 is replaced may be avoided, and thus the dressing is convenient for user. Moreover, although the embodiments above illustrate one barrier layer 124, the invention is not limited thereto. In other words, in other embodiments, the composite substrate 120 may also include a plurality of stacked barrier layers 124, wherein the barrier layers 124 may have the same or different materials.

The gel layer 130 is disposed on the composite substrate 120, wherein the substrate layer 122 is located between the gel layer 130 and the barrier layer 124. The barrier layer 124 prevents liquid from permeating from the substrate layer 122 to the surface 110a of the back layer 110. The gel layer 130 includes, for instance, a hydrogel layer, and contacts the wound. The forming method of the gel layer 130 is, for instance, coating the substrate layer 122 of the composite substrate 120 with a mixed solution including the unreacted main material, and then solidifying the mixed solution by photopolymerization or thermal polymerization to form the gel layer 130. In particular, the unreacted main material includes a plurality of chemical ingredients such as polymerizable monomers, crosslinking agents, and initiators. The chemical ingredients include alkenes, amines, alcohol, amides, the derivatives thereof, or combinations thereof, and are usually irritating to the skin. Moreover, the mixed solution including the unreacted main material is usually a runny liquid, the fluidity of which changes with different materials or ratios, and therefore the reaction conditions and the reaction time are also changed. In general, after coating the substrate layer 122 with the mixed solution, the unreacted main material is embedded in the cavities of the substrate layer 122 to facilitate physical adhesion and polymerization at the same time in order to form the gel layer 130. In the present embodiment, since the barrier layer 124 has the functions of support and preventing liquid from permeating, factors such as gravity, external forces, or different reaction rates do not cause the unreacted material to permeate to the back of the composite substrate 120 (the side joining the back layer 110), which facilitates the main material to react completely in order to form the gel layer 130 having a uniform thickness. In this way, the unreacted main material does not diffuse through the bottom of the substrate layer to the reacted main material over time. Therefore, skin irritation, allergy, or other conditions in the wound are avoided when the dressing contacts the skin. Moreover, the thickness of the layer formed by coating the mixed solution may easily be controlled, so that the gel layer 130 has a desired thickness.

In summary, the invention provides a dressing having a composite substrate, wherein the composite substrate includes a substrate layer and a barrier layer, and the barrier layer may prevent liquid from permeating from the substrate layer to the back of the composite substrate (the surface of the back layer). In this way, permeation of the solution to the surface of the back layer may be avoided, and thus the ingredients in the solution can be reacted completely to form a gel layer with a uniform thickness. Accordingly, the adhesion between the composite substrate and the back layer is improved. Therefore, the safety, the stability, and the convenience of use of the dressing are significantly improved.

Although the invention has been described with reference to the embodiments thereof, it will be apparent to one of the ordinary skills in the art that modifications to the described embodiments may be made without departing from the spirit of the invention. Accordingly, the scope of the invention will be defined by the attached claims not by the above detailed description.

What is claimed is:

1. A dressing, comprising:
a back layer having a surface;
a composite substrate disposed on the surface of the back layer, comprising:
a substrate layer; and
at least one barrier layer disposed between the back layer and the substrate layer; and
a gel layer disposed on the composite substrate, wherein the substrate layer is located between the gel layer and the barrier layer, and the barrier layer prevents liquid from permeating from the substrate layer to the surface of the back layer.

2. The dressing of claim 1, wherein the composite substrate comprises an adhesive layer disposed between the barrier layer and the substrate layer.

3. The dressing of claim 2, wherein the adhesive layer comprises a hot melt adhesive, a pressure sensitive adhesive, or an acrylic glue.

4. The dressing of claim 1, wherein the barrier layer comprises a lamination layer.

5. The dressing of claim 1, wherein the gel layer comprises a hydrogel layer.

6. The dressing of claim 1, wherein the substrate layer comprises a porous membrane.

7. The dressing of claim 1, wherein a material of the substrate layer comprises polyethylene, polypropylene, polyester, polycarbonate, polyurethanes, derivatives thereof, or a combination thereof.

8. The dressing of claim 1, wherein the barrier layer comprises a non-porous membrane.

9. The dressing of claim 1, wherein a material of the barrier layer comprises polyethylene, polypropylene, polyester, polycarbonate, polyurethanes, derivatives thereof, or a combination thereof.
